(19) United States
(12) Patent Application Publication
(10) Pub. No.: US 2004/0171956 A1
(43) Pub. Date: Sep. 2, 2004

(54) Heart rate monitor using color to convey information

(76) Inventor: Bruce Babashan, Bethesda, MD (US)

Correspondence Address: Richard C. Litman, LITMAN LAW OFFICES, LTD., P.O. Box 15035, Arlington, VA, 22215, US

(21) Appl. No.: 10/761,218

(22) Filed: Jan. 22, 2004

Related U.S. Application Data
(60) Provisional application No. 60/443,567, filed on Jan. 30, 2003.

(51) Int. Cl. A61B 5/0402

(52) U.S. Cl. 600/509

(57) ABSTRACT

A heart rate monitor provides a display of the user's heart rate range or zone, by displaying a color homogeneously and uniformly across a display field on the device. The heart rate monitor is thus much more easily read and interpreted by a person engaged in vigorous exercise than monitors having relatively small digital numerical displays. The colors may be keyed mnemonically to assist the user in interpreting the display, and/or the user may program the device to display specific colors according to the level of the user. Certain variables, e.g., age and gender of the user, may be input as desired in order to provide greater accuracy for the device. The present heart rate monitor may be configured as a portable device to be worn by the user, or as a device installed with a stationary exercise machine, e.g. treadmill, etc.

### CROSS-REFERENCE TO RELATED APPLICATION

This application claims the benefit of U.S. Provisional Patent Application Serial No. 60/443,567, filed Jan. 30, 2003.

### BACKGROUND OF THE INVENTION

1. Field of the Invention

The present invention relates generally to heart and pulse rate reporting devices used in exercise and fitness training and programs. More specifically, the present invention comprises a heart rate monitor which provides the user with a color field to indicate the general range of heart rate being achieved, rather than solely a digital numerical readout.

2. Description of Related Art

It has been recognized for some time that the degree of elevation of the heart rate during exercise is an indication of the level of exercise being performed. More recently, studies have determined that the greatest benefit from exercise is achieved when the exercise is performed to elevate the heart rate to a specific predetermined range, and held in that range for the duration of the exercise. More specifically, it is desired that the heart rate be raised gradually into the desired range by a series of warm-up exercises, and allowed to drop back gradually to its normal rate by a series of cool down exercises. The greatest benefit to the person involved, and the least stress and strain on the heart, is achieved when exercises are performed according to this philosophy.

With the increasing popularity of various fitness training and exercise programs, more and more amateur and professional athletes are paying greater attention to specific heart rates achieved during exercise, as recommended by their trainers and other programs. Technology has resulted in the development of the heart rate monitor, comprising an electronic device which detects the pulse of the user and provides a readout of the user's pulse rate. Various principles have been developed for detecting the pulse of a person using such a device, e.g., the tonometer and oximetry principles, as well as invasive means which are impracticable in a heart rate monitor for exercising persons.

Greater interest in the subject by those in the medical field, has also resulted in the development of a number of different formulas for determining optimum heart rate for any given conditions or level of exertion. The Karvonen formula for determining optimum heart rate, is one such formula which has been known and used for some time by those who are knowledgeable in the field. The Karvonen formula determines a target heart rate by subtracting the exercising person's age and resting heart rate from an initial number, e.g., 220 (for men) or 226 (for women); other numbers may be used. The target range is typically in a range between 50 and 85 percent of the target heart rate, plus the resting heart rate. The target range may vary from this exemplary range, depending upon the specific exercise program being used. The Karvonen formula is well known, and is used by perhaps the great majority of exercise programs which specify target heart rates during exercise. Other formulas for approximating optimum heart rate during exercise have been developed, as well as stress tests. for determining heart rate.

While many heart rate monitors have been developed with digital pulse rate displays, with some of these monitors also providing indications of the optimum or target heart rate in accordance with the Karvonen or other formula, such displays have always been accomplished by digital means in the prior art. Such digital displays of heart rate, and/or target rates, do not provide for ease of reading the display under most conditions of use, where the user is exercising vigorously. As an example, when a user is jogging, relatively rapid arm movement along with the at least somewhat jarring motions produced by rapid impact of the feet with the running surface, can make it extremely difficult to read a relatively small digital display. This is all the more true in various other forms of exercise, e.g., rowing, calisthenics, etc., where arm motion does not position a wrist mounted device for reading a display thereon. Even when using stationary treadmill type devices, it can be difficult to read a relatively small digital display provided thereon. Moreover, it is not critical that an exercising person establish a precise heart rate, but rather that the exercise maintain the heart rate within a desired range, e.g. in accordance with the Karvonen formula and other formulas which approximate a desired heart rate range during exercise.

The present invention responds to this problem by providing a heart rate monitor which displays the general range of the user's heart rate, by means of a color display. The present invention comprises a display (either portable or permanently installed on an exercise device or the like, as desired) and user input means for setting basic variables e.g., user's age and gender) into the device. Other models may include means for inputting additional variables in various ways, as desired. The present heart rate monitor preferably provides an easily viewed field which displays a uniform color homogeneously across a substantial portion of the field, enabling a user of the device to tell at a brief glance, just which heart rate range or zone he or she is in at the moment. Different colors may signify different ranges, e.g., blue for cool down (or warm-up), red to indicate “fat burning,” black to indicate the “dead zone” for trained athletes who need to reach a higher level of cardiovascular activity, etc. In some models, additional input means may be provided to allow the user to adjust the color display as desired, depending upon the fitness level of the user and the type of activity to be performed.

A discussion of the related art of which the present inventor is aware, and its differences and distinctions from the present invention, is provided below.

U.S. Pat. No. 4,647,217 issued on Mar. 3, 1987 to Karel Havel, titled “Variable Color Digital Timepiece,” describes a watch with a digital display, in which the digits may be colored to indicate some additional condition. Havel provides a pulse sensing transducer, which he interfaces with the color control system for the digits of his display. Thus, the display digits may change color in accordance with the heart rate detected by the pulse sensing device. This is considerably different from the present invention, in that the Havel color display comprises a series of relatively small, individual digits, rather than a relatively large, homogeneous and uniform color field. A person using the Havel device would not likely be able to interpret the color indications of the display digits in a relatively small wristwatch form while moving or swinging his or her arm during vigorous exercise. Havel attempts to provide many different components of information in a single display. While this may be efficient in some circumstances, it also tends to make the information difficult for the user to interpret, due to the relative complexity of the display. Persons using heart rate monitors must be able to read them under difficult conditions, as during vigorous exercise, and the relatively small resolution of the Havel digital display and its colors do not appear to meet these requirements to the degree necessary. Moreover, Havel fails to provide any form of user input to adjust or set various parameters or variables, such as the age and gender of the user. The Havel color indications for heart rate, are thus not particularly useful to persons having physical characteristics which differ from the single model from which the Havel display was programmed. The present heart rate monitor provides for such user input, in order to provide a more specific and meaningful display.

U.S. Pat. No. 5,000,188 issued on Mar. 19, 1991 to Osamu Kojima, titled “Physiological Age Measuring Apparatus,” describes a pulse wave sensor and corresponding equipment and programming, enabling the user to determine the physiological age of the subject or patient. The device can detect minute variations in the pulse waveform, and thus determine the degree of arteriosclerosis (hardening) present in the subject, which arteriosclerosis corresponds to the physiological age of the subject. Kojima notes two exemplary means of detecting the pulse of the subject, i.e., piezoelectric means or a semiconductor type strain gauge. Such devices are well known in the art, and may be used in the present pulse rate monitor in lieu of the tonometer and oximetry principles noted further above. However, Kojima does not disclose any means for measuring the frequency of the pulse waveform detected by his apparatus. Pulse rate is defined as a series of individual pulses or beats divided by a time interval, generally noted as beats per minute. The Kojima device is not a heart rate monitor, as Kojima does not disclose any timer means or function in his apparatus. Moreover, Kojima does not disclose the use of a color display to indicate a range or zone of heart beat rate, as provided by the present heart rate monitor.

U.S. Pat. No. 5,197,489 issued on Mar. 30, 1993 to Robert W. Conlan, titled “Activity Monitoring Apparatus With Configurable Filters,” describes a device which detects the frequency of physiological movements, including pulse. The device is relatively small and portable, and may be worn upon the wrist of a user. However, the Conlan device is configured to record information, and upload that information to a computer for later processing as desired. Accordingly, Conlan does not provide any form of display means in his device. Thus, a person using the Conlan device would not be able to determine their pulse rate, or whether or not their pulse rate is within the desired range, during an exercise period in real time when such information is needed by the exercising person.

U.S. Pat. No. 5,243,992 issued on Sep. 14, 1993 to Joseph S. Eckerle et al., titled “Pulse Rate Sensor System,” describes a small, portable pulse rate monitor or indicator for wearing upon the wrist. The Eckerle et al. device uses the tonometer principle of pulse detection, and provides a digital readout or display of the pulse rate of the wearer. The Eckerle et al. device also provides an alarm to indicate when an excessively high or low pulse rate has been reached by the user. The Eckerle et al. pulse rate sensor with its digital display is essentially conventional in view of other devices known to the present inventor, and does not provide a color field indication of pulse rate, as provided by the present invention. Moreover, Eckerle et al. do not appear to provide any means for the user to set his or her age, sex, and/or other factors in their device. Thus, any alarms for excessively high or low heart rates would have to be excessively broad so as to avoid false alarms for most users, or would tend to create false alarms if set to too narrow a range.

U.S. Pat. No. 5,431,170 issued on Jul. 11, 1995 to Geoffrey R. Mathews, titled “Pulse Responsive Device,” describes a pulse rate monitor using a light detection principle (oximetry) device for detecting the pulse of the individual using the device. Mathews also provides for user input of age and gender in order to “customize” the resulting output to a greater degree, and also provides a display of the desired upper and lower limits for a given cardiovascular activity. However, the Mathews display is a conventional, monochromatic digital display, and does not provide any form of color display. The disadvantages of such digital displays, whether monochromatic or including color, have been noted further above.

U.S. Pat. No. 5,529,073 issued on Jun. 25, 1996 to Peter Kielbasiewicz, titled “Method And Apparatus For Recording Physiologic Signals,” describes a dual heart monitoring system for measuring the heart rate of twin fetuses in the womb. Kielbasiewicz provides an offset mode for one of the signals, in order to make the signals more distinct from one another when pulses are occurring simultaneously. Kielbasiewicz does not provide any form of color output for a desired pulse rate during exercise nor does he provide any user adjustable input, as obviously such functions are meaningless in the environment wherein the device is used to measure the heart beats of fetuses.

U.S. Pat. No. 5,539,706 issued on Jul. 23, 1996 to Masaaki Takenaka et al., titled “Pulsimeter Provided With Or Without A Pedometer,” describes a combination pulse rate monitor and pedometer, with the pulse rate detector comprising an oximetric type device. Takenaka et al. provide only a monochromatic, digital pulse rate display, with no color or color field being disclosed. As the Takenaka et al. device is relatively small due to its configuration for fitting upon the finger of the user, the display is so small that it is not readily legible by the user during vigorous exercise. Moreover, Takenaka et al. do not provide any user input means for entering the age, sex, and/or other user variables which may be relevant.

U.S. Pat. No. 5,558,096 issued on Sep. 24, 1996 to Eugene S. Palatnik, titled “Blood Pulse Detection Method Using Autocorrelation,” describes a medical monitoring device using the oximetry principle of pulse detection. As the Palatnik device is intended for use as a medical monitor, no user adjustable inputs are provided, nor is any form of user readable display disclosed by Palatnik.

U.S. Pat. No. 5,807,267 issued on Sep. 15, 1998 to John D. Bryars et al., titled “Heart Pulse Monitor,” describes a small, portable heart rate monitor for wearing upon the wrist of the user. The device has a similar configuration to a conventional digital wristwatch, and in fact displays the time and date as well as the pulse rate of the user. However, the pulse display (and time and date, as well) are displayed digitally. Bryars et al. make no disclosure of the use of color in their monitor. Moreover, no user input of variables is provided by Bryars et al. in their monitor. The only indicator provided for any variable, is a small heart-shaped pattern which indicates the signal strength provided from the pulse sensor of the device. This does not provide any indication of maximum, minimum, or optimum desired heart rates.

U.S. Pat. No. 6,447,458 issued on Sep. 10, 2002 to Robert M. Farrell et al., titled “Method And System Of Color Coding Components Of Central Venous And Pulmonary Artery Wedge Pressure Waveforms,” describes a medical process using an invasive catheter as the pulse sensing device. This method is completely unsuitable for the athlete or other user who is not working or exercising in a medical environment. As the Farrell et al. device is intended to be used in a medical environment, there is neither input nor display available to the person actually using the device, i.e., the patient or subject being monitored by the system. The only color coding disclosed by Farrell et al. is provided by a computer program which shades certain portions of the pulse waveform as it is graphed. No optimum pulse rate as indicated by a color display, is provided by the Farrell et al. device.

U.S. Pat. Publication No. 2001/16,689 published on Aug. 23, 2001 to Ilkka Heikkila et al., titled “Measurement Relating To Human Body,” describes a neural network system for processing the pulse rate detected by a user of the device. While Heikkila et al. disclose several factors which may be considered by their system, they do not disclose any form of color display indication of pulse rate for the user who is being monitored by the system.

U.S. Pat. Publication No. 2002/55,418 published on May 9, 2002 to Nathan Pyles et al., titled “Interactive Fitness Equipment,” describes a relatively complex and cumbersome system which permits an exercising person (e.g., on a treadmill or stationary exercise bike, etc.) to access a computer while exercising. While Pyles et al. can provide a display of the exercising person's heart rate. on the computer screen during the exercise period, they do not provide any form of color display of the heart rate range, nor any means for the user to input his or her age and gender, as provided by the present heart rate monitor. Moreover, the Pyles et al. device is clearly not portable and cannot be carried conveniently upon the wrist or other area of the body of the user, as is possible with at least one embodiment of the present invention.

Finally, U.S. Pat. Publication No. 2002/120,201 published on Aug. 29, 2002 to Shiu-Shin Chio et al., titled “Hemodynamic Analysis Device And Method,” describes a cardiovascular monitoring system for transmitting cardiovascular data from a patient to a remote site, via the internet. No means is provided for inputting criteria from the patient or person being monitored by the Chio et al. device, and no disclosure is made by Chio et al. of any form of color display field to show an optimum heart rate zone or range for the user.

None of the above inventions and patents, taken either singly or in combination, is seen to describe the instant invention as claimed.

### SUMMARY OF THE INVENTION

The present invention comprises various embodiments of a heart rate monitor which provides information on the heart rate of the user in the form of a relatively large color field to indicate a general range or zone for the user's heart rate. This means of conveying heart rate information is a considerable improvement over digital displays used in the past, as the user is able to determine at a glance whether or not his or her heart rate is in the desired range. The relatively small digital displays conventionally used for providing heart rate information in a heart rate monitor are quite difficult to interpret during vigorous exercise, particularly in the case of small, wrist attached heart rate monitors when the user is moving or swinging his or her arms vigorously. Even in the case of stationary, permanently installed monitors used with exercise bicycles, rowing machines, treadmills, etc., the conventional digital displays can be difficult to read, due to the movement of the person using the device. Moreover, even in those cases where the display can be read by the user, there is little point in providing heart rate information to the resolution generally achieved by such devices, i.e. displaying the pulse rate to the nearest single beat per minute during vigorous exercise. Not only are such devices difficult to read during vigorous exercise, but the user must also calculate the desired heart rate range or zone for the exercise being accomplished, and consider whether or not the displayed heart rate number is within this zone or range.

The present heart rate monitor responds to these problems by providing a color display which indicates a general range or zone for the heart rate, rather than a specific number. The present heart rate monitor may be configured in a relatively small, portable embodiment for wearing upon the wrist of the user or for carrying in the hand of the user, or may comprise a permanently installed device incorporated with a stationary exercise machine or other apparatus, as desired. The common theme between each of the embodiments of the present invention, is the provision of an easily viewed field which displays a uniform color homogeneously on a substantial portion thereof. The color displayed corresponds to a heart or pulse rate range, rather than to a specific number. The person using the present heart rate monitor, need only exercise as required to cause his or her heart rate to reach the desired zone, whereupon the color field will indicate such by displaying the appropriate color. Input means is provided with the device, enabling the user to input variables such as his or her age and gender, and/or perhaps other variables as well, depending upon the degree of complexity desired for the device.

An algorithm is programmed into the device to control the color field display in accordance with the heart rate range or zone achieved by the user. The specific algorithm or formula is not particularly critical to the function of the present invention; any one of several known algorithms, or such algorithms as may be developed in the future, may be programmed as desired into the microcontroller of the present heart rate monitor. An example of such an algorithm is the Karvonen formula, which determines a target heart rate by subtracting the exercising person's age and resting heart rate from e.g. 220 (for men) or 226 (for women). The target range is between 50 and 85 percent of the target heart rate, plus the resting heart rate. The present heart rate monitor includes means for the user to input his or her age in order to use the Karvonen algorithm as described above. Other variables, such as the user's sex, and perhaps other factors, may be input as well, depending upon the complexity of the specific embodiment of the present heart rate monitor and the algorithm or formula programmed therein.

Means may be provided to record heart rate information over the duration of an exercise period, and download the recorded information to a computer, if so desired. The microcontroller used in the present heart rate monitor may also be programmed to provide estimates of other functions, such as calories burned during a workout, etc. The display field may include a digital time display superimposed over the color display and independent thereof, enabling the device to be used as a wristwatch, stopwatch, or timepiece if so desired. As such a digital time indication may be difficult to read during exercise, the device may indicate in some other manner, e.g. by flashing the color field display, that a predetermined exercise period or duration has been reached. Other conventional features, e.g., battery saver mode, etc., may be incorporated into the present heart rate monitor as desired. It will also be seen that the present color display field may be incorporated into other devices as well, such as depth gauges for scuba divers, altimeters for skydivers, etc., where a quickly readable display is critical.

The provision of an easily viewed color display field in the present heart rate monitor, also provides considerably greater versatility for its use. For example, the present heart rate monitor is not limited only to use with humans who desire to have an easily interpreted view of the range of their heart rates. The present heart rate monitor in its portable configuration is also readily adaptable to use with, animals. As an example, the present heart rate monitor may be applied to a race horse during exercise periods. The trainer or rider can easily see the color field display provided by the present heart rate monitor, and exercise the animal accordingly to achieve the desired color display, and thus the desired heart rate which corresponds to the desired level of exertion. The present heart rate monitor in its portable form is sufficiently small to be placed upon smaller animals as well (e.g., greyhounds, etc.), yet the easily viewed display permits a trainer to note the heart rate range of the animal from some distance away.

### BRIEF DESCRIPTION OF THE DRAWINGS

FIG. 1 is a block diagram of the basic components and inputs thereto for the heart rate monitor of the present invention.

FIG. 2 is an environmental top plan view of a first embodiment of the present heart rate monitor being worn upon the wrist of a user, showing the basic external features of the device.

FIG. 3 is a detailed top plan view of the heart rate monitor of FIG. 2, illustrating an exemplary device for inputting the age of the user to the device.

FIG. 4 is a top plan view of the heart rate monitor of FIG. 3 with the display removed, illustrating an exemplary internal mechanism for inputting a variable to the microcontroller of the device.

FIG. 5 is a perspective view of a stationary treadmill exercise device incorporating an alternative embodiment of the present heart rate monitor therewith.

Similar reference characters denote corresponding features consistently throughout the attached drawings.

### DETAILED DESCRIPTION OF THE PREFERRED EMBODIMENTS

The present invention comprises a series of embodiments of a heart rate monitor having a large color display field for indicating the heart beat frequency range of a user of the device. The present heart rate monitor may utilize conventional technology to detect the heart beat or pulse of a user, and may be constructed as a relatively small and portable device worn on the wrist or other area of the body or face (e.g., sunglasses) of the user, or as a larger device temporarily or permanently installed in a stationary exercise machine (e.g., treadmill, rowing machine, etc.).

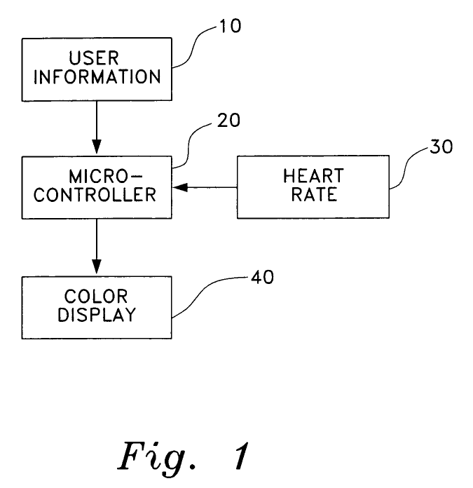

FIG. 1 of the drawings provides an illustration of the basic components of the present invention, and their relationship to one another. The central component of the present invention is a microcontroller 20, which receives input from two sources, i.e., a conventional transducer or input device 30 which measures the heart rate of the user, and a user input device 10. The microcontroller 20 then processes this information and controls an easily viewed color display field 40, with the color displayed being in accordance with the heart rate measured by the heart rate transducer 30.

The microcontroller 20 is conventional, with various such devices being available in the marketplace for carrying out the required functions of the present invention, i.e., measuring a pulse frequency and controlling a color display in accordance with the frequency detected. The inventive concept of the present invention comprises the use of an easily viewed color display to indicate a general range of heartbeat or pulse frequency. The microcontroller may be configured to interface with various computer devices, e.g., a personal digital assistant (PDA) device, etc., in order to record information from the present invention for later review. The microcontroller 20 is programmed with any one of a number of known formulas or algorithms for determining the optimum heart rate of a person during exercise. In the example cited herein, the Karvonen formula is used.

The Karvonen formula comprises the calculation of a target heart rate, from which a heart rate reserve range is calculated. A. constant is initially provided, with the constant being different for men and women. For men, this constant is generally set at 220, and for women, 226. The present heart rate monitor invention may provide for user input for the sex or gender of the user, in order to provide the proper constant. Once the constant has been determined, the user subtracts his or her age and his or her resting heart rate from the constant, to provide a base heart rate number from which maximum and minimum heart rates during exercise are calculated. The respective maximum and minimum heart rates are generally eighty five percent and fifty percent of the base number, plus the resting heart rate.

As an example of the above, a thirty year old male with a resting heart rate of seventy, would subtract his age and resting heart rate from the initial constant, i.e., 220−30−70=120. The person would then multiply this result (120) by fifty percent and eighty five percent and add his resting heart rate to each result, to arrive at his respective lower and upper desired heart rates during exercise. Thus, the lower heart rate limit would be (120×0.5)+70=130, and the upper heart rate limit would be (120×0.85)+70=172. The microcontroller 20 of the present heart rate monitor automatically calculates the above numbers, once the user has entered his age and gender into the device. The resting heart rate of the user is determined automatically by the heart rate transducer 30.

The heart rate transducer or input device 30 may comprise any of a number of known devices and/or principles of operation. A basic means of electronically detecting heart or pulse rate was developed by Willem Einthoven in 1906, with many pulse rate detectors using the same principle of operation today. Other principles and devices, e.g., plethysmography using an optoelectronic transducer, Doppler ultrasonography using a piezoelectric transducer, etc., may be used as desired for the heart rate transducer 30.

Once the microcontroller 20 has received the appropriate heart rate signals from the heart rate input transducer 30, the microcontroller 20 then provides an appropriate signal to the color display field 40. The color display 40 displays a color in accordance with the heart rate frequency detected by the heart rate transducer 30, as processed by the microcontroller 20 according to the algorithm or formula programmed therein. The optimum display is a color display disposed uniformly and homogeneously over a substantial portion of the color display field 40 to provide an easily viewed and interpreted indication of the corresponding general heart rate range of the user. The use of an easily viewed color field 40 allows a user of the present device to determine his or her general heart rate range at a glance without needing to stop the exercise for a short period of time in order to read and interpret a relatively small digital display, as is conventionally provided with heart rate monitors.

Examples of the colors and corresponding heart rate ranges with which the present heart rate monitor might be programmed are provided below. In accordance with the exemplary Karvonen formula described further above, the user of the present device desires to maintain his or her heart rate within some predetermined range, e.g., between fifty and eighty five percent of the base heart rate number. The user begins an exercise session with a warm-up period, during which the body is warmed up relatively slowly, muscle groups are stretched, and the heart rate slowly increases. This relatively “cool” exercise zone, comprising a heart rate between fifty and sixty percent of the base heart rate number, may be programmed to provide a blue color or tint distributed homogeneously and uniformly over a substantial portion of the color display field 40. Thus, the exercising person using the present heart rate monitor need only glance at the display 40 to determine whether or not he or she is working at the desired level. Once the relatively cool “warm-up” period has been completed, the exercising person may exert himself or herself somewhat more strenuously, thus elevating the heart rate to a somewhat higher level. The desired heart rate during this period may be between sixty and seventy percent of the base heart rate number, and may result in a green heart rate display field 40 to indicate a desired level of performance or exertion.

In many instances, the exercising person may wish to reach a higher, anaerobic exercise state or level, in which the muscle groups are exercised more strenuously and the heart rate is increased correspondingly. This heart rate level may be between seventy and eighty percent of the previously calculated base heart rate, and may result in a red color being displayed on the color display area 40, to indicate a “fat burning” exercise level. Even higher levels of exercise may result in other colors, e.g., a yellow or “caution” range for a heart rate between eighty and ninety percent of the base heart rate, and black when the heart rate exceeds ninety percent of the base rate. These colors are exemplary, and other colors may be programmed into the device as desired. For example, a trained marathon runner may exert himself or herself to a reasonable level with a relatively low heart rate, and not develop his or her abilities further. This level of exercise is called the “dead zone” by many trainers and advanced athletes, as it does not provide the level of physical training they desire. The present heart rate monitor may be programmed to provide a black display when this level is reached, if so desired.

The display field 40, with its easily viewed and interpreted color display, enables an exercising person to note whether he or she is in the proper activity range, even though considerable body movement is likely occurring which would preclude the ability to read a small digital display. Persons who normally wear corrective lenses, but remove them for exercise, will find the present monitor to be particularly useful. Also, the ability to program the device to provide different colors in the display for different heart rate activity levels, also provides for those persons who may have some degree of color blindness. A common form of color blindness is difficulty in distinguishing red and green. Accordingly, different colors may be used, e.g., blues, yellows, and/or perhaps oranges or other colors somewhat removed from the center of the red area of the spectrum, etc., as desired. In addition, further information may be provided by pulsing or flashing the display to attract the user's attention and/or to indicate some other condition or information.

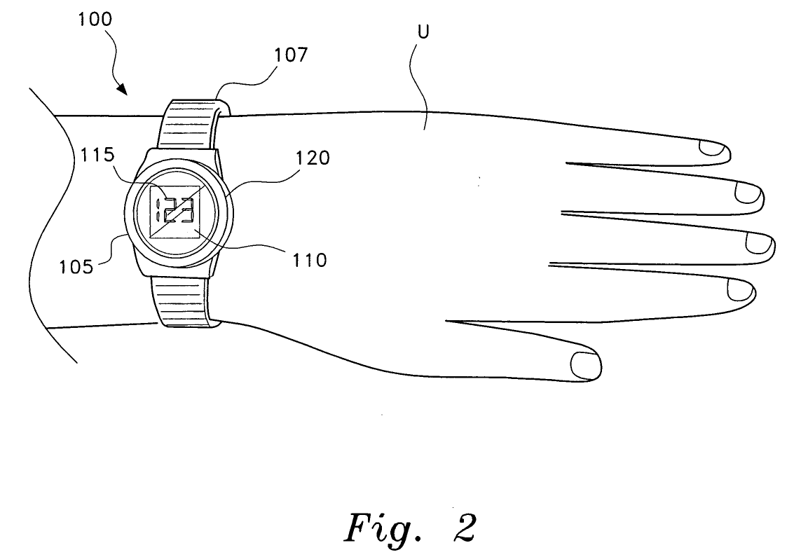

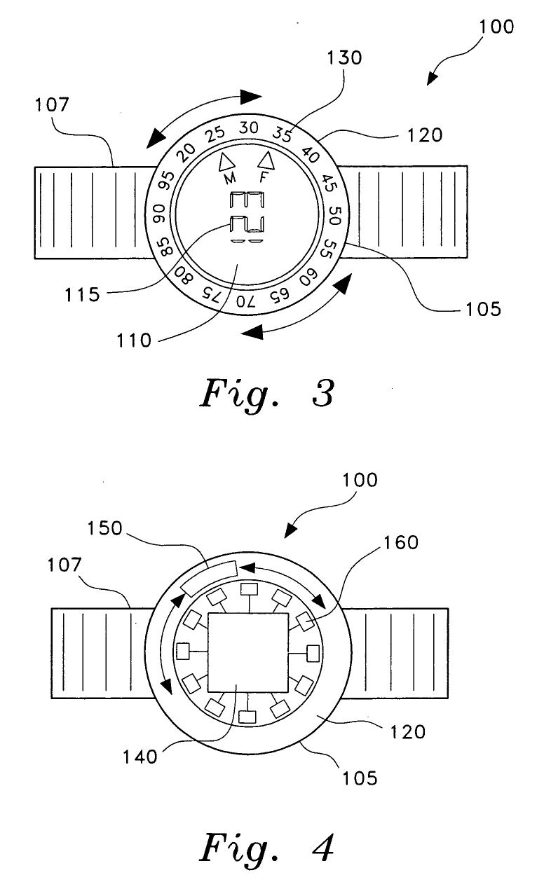

FIGS. 2 and 3 of the drawings provide top plan views of one embodiment of the present heart rate monitor invention, comprising a wrist mounted or attached heart rate monitor device 100, similar in configuration to a conventional wristwatch. The wrist mounted monitor 100 includes a housing or case 105, with a wrist strap 107 extending from each side thereof for conventional attachment of the device 100 to the wrist of a user U. The case 105 contains the various componentry shown in the flow chart of FIG. 1, i.e., the microcontroller 20 and heart rate transducer 30. Alternatively, the transducer 30 may be located along the wrist band 107 or elsewhere on the body, with suitable communication between the transducer 30 and microcontroller 20 being provided.

The easily viewed color display field 110 is disposed upon the outer surface of the case or housing 105, where it is clearly visible to the user U wearing the wrist mounted monitor 100. The color display field 110 preferably encompasses the majority of the face of the case or housing 105, in order to provide the desired color surface area for ease of viewing by the user U. Various means of providing the uniform color display desired in the present heart rate monitor invention, may be used. For example, where relatively high electrical power consumption is not a concern, a matrix or array of pixels as used in flat screen television screens, or light emitting diodes (LEDs), may be used as desired. The technology also exists to provide color in a liquid crystal display, particularly by incorporating a stacked array to provide spectral diffraction to produce the desired color effects. Reflective LCD displays are also possible, and require less electrical power than do the other technologies noted above. Alternatively, an electromechanical display may be constructed, utilizing a small display band having the desired display colors applied to various areas thereof. The band may be rolled from end to end, with the exposed central area passing beneath the window of the display field 110. Movement of the band may be accomplished by micro-size electrical motors, or more economically by small solenoids which actuate an escapement mechanism at each roller. This system requires no electrical power whatsoever when the band is stationary.

The forming of the color display field 110 from a large number of relatively small elements, generally as described above, enables the programming to change the color, shading, or brightness displayed upon some of the elements to contrast with the remainder of the color field. Thus, a supplementary message may be superimposed upon the primary uniform color display field, if so desired. Such a supplementary message may be in the form of a digital display 115, as indicated in FIGS. 2 and 3, or some other display format, as desired. It is not intended that such a digital display provide crucial information relating to heart rate during an exercise period. This function is accomplished by the easily viewed color display field 110. In fact, the digital display 115 is not required with the present heart rate monitor, but may be provided optionally if so desired. The digital display 115 may provide the time, or perhaps a time interval for the exercise session or portion thereof, or an estimate of calories burned, etc., as desired. Conventional controls, e.g. a rotating stem or button (not shown) as used to set and adjust the time in conventional wrist watches, may be provided to adjust, activate, and/or deactivate the digital display 115 as desired.

All formulas or algorithms used for determining the optimum heart rate of an exercising person require the input of certain variables which are dependent upon characteristics of the exercising person. Such variables may comprise the person's age, sex, height and weight, and fitness level, and/or other parameters. For example, the Karvonen formula takes into account a person's age and gender, as well as his or her resting heart rate. The resting heart rate may be determined automatically by the present heart rate monitor, as noted further above. However, the other parameters must be entered into the device by the user. Accordingly, a user input device 120 is provided in the wrist mounted heart rate monitor 100 of FIGS. 2 and 3. The user input device 120 comprises a rotating bezel which surrounds the display area 110, and generally defines the circumference of the case or housing 105. The bezel 120 preferably includes a series of numbers 130 thereon which correspond to the age of the user, and separate index marks for males and females to accommodate their different initial constants.

A person using the present heart rate monitor 100 of FIGS. 2 and 3, need only rotate the user input bezel ring 120 to align the appropriate age number 130 thereon, with the corresponding index mark “M” (males) or “F” (females), as appropriate. The device automatically detects the person's resting heart rate when the device is worn while the user is at rest. This is all the information needed for the device 100 to calculate the various heart rate ranges desired during exercise for the person using the present device 100, in accordance with the Karvonen formula. Alternative formulas or algorithms which take into account other factors may be programmed into the present device in lieu of the Karvonen formula if so desired, with the user input controls being marked and indexed accordingly. It will be seen that other means of entering user variables, e.g., a series of pushbuttons, rotary knobs, etc., may be incorporated with the present device, if so desired. Such setting and adjustment buttons and knobs are conventional, and are well known in the field of controls for miniaturized equipment.

FIG. 4 is an illustration of the internal configuration of the present wrist mounted heart rate monitor 100, showing an exemplary electrical contact system for programming the microcontroller 140 contained therein. The internal volume of the case 105 contains a plurality of electrical contacts 160 therein, disposed in a radial array immediately inside the circumference of the case 105. These electrical contacts 160 communicate electrically with the microcontroller 140 disposed within the case 105. An electrical resistor 150 is disposed within the ring comprising the rotating user input bezel 120. As the user rotates the bezel 120, the resistor 150 comes into electrical contact with different ones or pairs of the electrical contacts 160 within the case or housing 105, thereby providing a signal(s) to the microcontroller 140 as to the appropriate age and sex or gender of the exercising person to be used for calculating the base heart rate of the user and the corresponding calculations of the desired heart rate ranges for that user during exercise. The color output of the display area 110 is adjusted accordingly during exercise, as described further above.

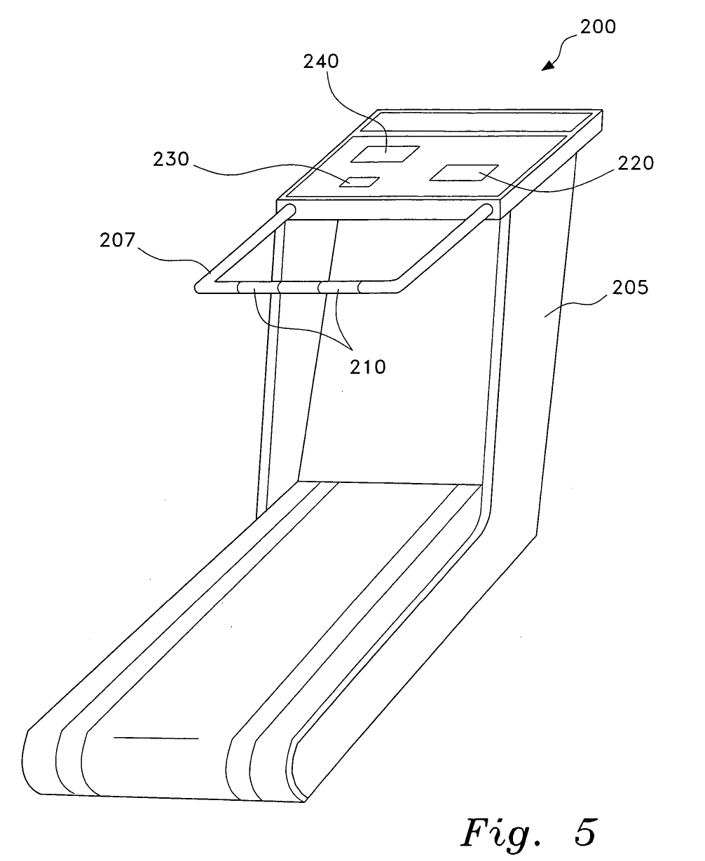

FIG. 5 provides a perspective view of an alternative installation of the present heart rate monitor device, wherein the device is permanently installed within a stationary exercise machine. The exercise machine illustrated in FIG. 5 comprises a treadmill 200, but it will be seen that the present heart rate monitor invention may be incorporated with virtually any type of stationary exercise equipment, e.g., rowing machines, exercise bicycles, weight machines, etc., as desired. The treadmill exercise machine 200 of FIG. 5 includes a stand 205 having various input controls and displays thereon. A handlebar 207 extends from the stand 205, with the handlebar 207 providing support for the user as well as a pair of handgrips 210 which include conventional heart rate transducer devices therewith. Other body contact means incorporating heart rate transducer devices may be incorporated as desired. The heart rate of the person using the exercise machine 200 is received by the handgrips 210, and transmitted to the microcontroller (not shown, but essentially the same as that used in the embodiment of FIGS. 2 through 4) for processing of the signal.

The stand 205 includes a conventional display 240 indicating distance covered and which may display additional information, e.g., estimated calories burned, etc. A conventional keypad 230 may be provided for the user to input information (user variables, etc.) as desired. The keypad 230 may be used to enter the exercising person's age, gender, and resting heart rate, as well as other information, e.g., height and weight, etc., as required by the particular program or formula being used with the machine 200. An easily viewed color display field 220 is also provided, with the display 220 being driven by the microcontroller (not shown) according to the programming of the microcontroller, the data entered using the keypad 230, and the heart rate of the user as detected by the handgrip transducers 210. The display 220 of the exercise machine 200 may utilize the same technology as described further above for the wrist attached heart rate monitor device 100, depicted generally in FIGS. 2 through 4. As the exercise machine 200 is stationary and receives electrical power from a remote source (e.g., 115 or 230 volt ac electrical power), the power consumption of some of the technologies noted, e.g., LEDs and backlighted displays, is not a concern.

In conclusion, the present heart rate monitor in any of its embodiments enables the user to quickly and easily note the general range of his or her heart rate while exercising. The easily viewed color display enables an exercising person to determine the level of their heart rate at a glance, without having to slow or stop the exercise activity to read and interpret a relatively small digital display, as is conventionally found in other heart rate indicating devices. The present heart rate monitor will also be beneficial to those persons who require corrective lenses, but who do not wear them during exercise. The easily viewed color display of the present heart rate monitor enables those persons with less than perfect eyesight, to note their general heart rate without need for any supplemental vision correction while exercising. The ease of comprehension of the present heart rate monitor will enable users to make better progress toward achieving their goals of better fitness and weight loss. As the colors provided by the display of the present heart rate monitor relate directly to established nomenclature and exertion levels, increased motivation and feedback is provided for users to enable them to improve their performance and achieve their goals. As the primary information required of most persons while exercising is their general heart rate range, and the knowledge that their heart rate (and thus their level of exertion) is appropriate for their condition, the present heart rate monitor in any of its embodiments will prove to be most beneficial to the average person who wishes to maintain their health.

It is to be understood that the present invention is not limited to the embodiments described above, but encompasses any and all embodiments within the scope of the following claims.

I claim:

1. A heart rate monitor, comprising:
a housing;
a microcontroller having a heart rate algorithm programmed therein disposed within said housing;
a heart rate input device communicating with said microcontroller; and
a heart rate color display field disposed upon said housing, displaying one of a plurality of colors homogeneously and uniformly over said color display field according to signals received from said microcontroller and according to heart rate input processed by said microcontroller from said heart rate input device.

2. The heart rate monitor according to claim 1, further including a user variable input device disposed upon said housing and communicating with said microcontroller.

3. The heart rate monitor according to claim 2, wherein said user variable input device is configured for at least one user variable selected from the group consisting of age, gender, height, weight, and fitness activity level.

4. The heart rate monitor according to claim 2, wherein:
said housing comprises a case configured for wearing upon the wrist of a user;
said case further includes a wrist strap extending therefrom; and
said user variable input device comprises a rotating bezel disposed about said case.

5. The heart rate monitor according to claim 4, wherein:
said case includes a plurality of radially disposed electrical contacts communicating with said microcontroller; and
said rotating bezel includes an internal electrical contact, selectively communicating with said plurality of electrical contacts within said case.

6. The heart rate monitor according to claim 2, wherein:
said housing comprises a stand extending upwardly from a stationary exercise machine; and
said user variable input device comprises a keypad disposed upon said stand.

7. The heart rate monitor according to claim 1, wherein:
said microcontroller determines which of said plurality of colors is displayed upon said color display field in accordance with the Karvonen formula; and
said plurality of colors comprise blue corresponding to a heart rate range of from fifty to sixty percent of the base heart rate, green corresponding to a heart rate range of from sixty to seventy percent of the base heart rate, red corresponding to a heart rate range of from seventy to eighty percent of the base heart rate, yellow corresponding to a heart rate range of from eighty to ninety percent of the base heart rate, and black corresponding to a heart rate range of from ninety to one hundred percent of the base heart rate.

8. A heart rate monitor, comprising:
a case configured for wearing upon the wrist of a user;
said case further including a wrist strap extending therefrom;
a microcontroller having a heart rate algorithm programmed therein, disposed within said case;
a heart rate input device, communicating with said microcontroller; and
a heart rate color display field disposed upon said case, displaying one of a plurality of colors homogeneously and uniformly over said color display field according to signals received from said microcontroller and according to heart rate input processed by said microcontroller from said heart rate input device.

9. The heart rate monitor according to claim 8, further including a user variable input device disposed upon said case, and communicating with said microcontroller.

10. The heart rate monitor according to claim 9, wherein said user variable input device comprises a rotating bezel disposed about said case.

11. The heart rate monitor according to claim 10, wherein:
said case includes a plurality of radially disposed electrical contacts communicating with said microcontroller; and
said rotating bezel includes an internal resistor, selectively communicating with said plurality of electrical contacts within said case.

12. The heart rate monitor according to claim 9, wherein said user variable input device is configured for at least one user variable selected from the group consisting of age, gender, height, weight, and fitness activity level.

13. The heart rate monitor according to claim 8, wherein:
said microcontroller determines which of said plurality of colors is displayed upon said color display field in accordance with the Karvonen formula; and
said plurality of colors comprise blue corresponding to a heart rate range of from fifty to sixty percent of the base heart rate, green corresponding to a heart rate range of from sixty to seventy percent of the base heart rate, red corresponding to a heart rate range of from seventy to eighty percent of the base heart rate, yellow corresponding to a heart rate range of from eighty to ninety percent of the base heart rate, and black corresponding to a heart rate range of from ninety to one hundred percent of the base heart rate.

14. The heart rate monitor according to claim 8, further including a user variable digital display disposed over said color display field.

15. A heart rate monitor, comprising:
a stand extending upwardly from a stationary exercise machine;
a microcontroller having a heart rate algorithm programmed therein, disposed within said stand;
a heart rate input device, communicating with said microcontroller; and
a heart rate color display field disposed upon said stand, displaying one of a plurality of colors homogeneously and uniformly over said color display field according to signals received from said microcontroller and according to heart rate input processed by said microcontroller from said heart rate input device.

16. The heart rate monitor according to claim 15, further including a user variable input device disposed upon said stand and communicating with said microcontroller.

17. The heart rate monitor according to claim 16, wherein said user variable input device comprises a keypad disposed upon said stand.

18. The heart rate monitor according to claim 16, wherein said user variable input device is configured for at least one user variable selected from the group consisting of age, gender, height, weight, and fitness activity level.

19. The heart rate monitor according to claim 15, wherein:
said microcontroller determines which of said plurality of colors is displayed upon said color display field in accordance with the Karvonen formula; and
said plurality of colors comprise blue corresponding to a heart rate range of from fifty to sixty percent of the base heart rate, green corresponding to a heart rate range of from sixty to seventy percent of the base heart rate, red corresponding to a heart rate range of from seventy to eighty percent of the base heart rate, yellow corresponding to a heart rate range of from eighty to ninety percent of the base heart rate, and black corresponding to a heart rate range of from ninety to one hundred percent of the base heart rate.

20. The heart rate monitor according to claim 15, further including a user variable digital display disposed over said color display field.
